SEQUENCE LISTING (6 sequences)

SEQ ID NO: 1 - DNA, 327 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggaggaaactgaagagacatcctcacaagagtctgccgaagaggac

SEQ ID NO: 2 - protein, 109 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWPQQVVQKKPAQEETEETSSQESAEED

SEQ ID NO: 3 - DNA, 321 bp, Homo sapiens
atgagtgagtcgagctcgaagtccagccagcccttggcctccaagcaggaaaaggacggcactgagaagcggggccggggcaggccgcgcaagcagcctccggtgagtcccgggacagcgctggtagggagtcagaaggagcccagcgaagtgccaacacctaagagacctcggggccgaccaaagggaagcaaaaacaagggtgctgccaagacccggaaaaccaccacaactccaggaaggaaaccaaggggcagacccaaaaaactggagaaggaggaagaggagggcatctcgcaggagtcctcggaggaggagcag

SEQ ID NO: 4 - protein, 107 aa, Homo sapiens
MSESSSKSSQPLASKQEKDGTEKRGRGRPRKQPPVSPGTALVGSQKEPSEVPTPKRPRGRPKGSKNKGAAKTRKTTTTPGRKPRGRPKKLEKEEEEGISQESSEEEQ

SEQ ID NO: 5 - DNA, 288 bp, Homo sapiens
atgagtgagtcgagctcgaagtccagccagcccttggcctccaagcaggaaaaggacggcactgagaagcggggccggggcaggccgcgcaagcagcctccgaaggagcccagcgaagtgccaacacctaagagacctcggggccgaccaaagggaagcaaaaacaagggtgctgccaagacccggaaaaccaccacaactccaggaaggaaaccaaggggcagacccaaaaaactggagaaggaggaagaggagggcatctcgcaggagtcctcggaggaggagcag

SEQ ID NO: 6 - protein, 96 aa, Homo sapiens
MSESSSKSSQPLASKQEKDGTEKRGRGRPRKQPPKEPSEVPTPKRPRGRPKGSKNKGAAKTRKTTTTPGRKPRGRPKKLEKEEEEGISQESSEEEQ

(19) United States
(12) Patent Application Publication
(10) Pub. No.: US 2010/0323354 A1
(43) Pub. Date: Dec. 23, 2010

(54) MEANS AND METHODS FOR THE DETECTION AND ISOLATION OF FETAL AND EMBRYONIC CELLS AND NUCLEIC ACID FROM MATERNAL BODY FLUID

(75) Inventor: Jörn Bullerdiek, Bremen (DE)

Correspondence Address: YAMAUCHI & MASON LLC, 60 Tufts Street, Suite #17, Somerville, MA, 02145, US

(73) Assignee: ALCEDO BIOTECH GMBH a, corporation

(21) Appl. No.: 12/740,477

(22) PCT Filed: Oct. 31, 2008
(86) PCT No.: PCT/EP2008/009218

§ 371 Date: Apr. 29, 2010

(30) Foreign Application Priority Data
Oct. 31, 2007 (EP) 07021314.5
Mar. 20, 2008 (EP) 08005389.5

(51) Int. Cl. C12Q 1/68; C12Q 1/02

(52) U.S. Cl. 435/6; 435/29

(57) ABSTRACT

The present invention is related to the use of an interaction partner of a high mobility group protein of the HMGA family for the detection of fetal and embryonic cells in a maternal body fluid.

The present invention is related to methods for the detection, separation and enrichment of fetal and embryonic cells as well as embryonic and/or fetal chromatin and/or fetal and/or embryonic nucleic acid(s) in/from a maternal body fluid, methods for the detection, separation and/or detection of tumor cells, and/or chromatin and/or nucleic acid(s) from a tumor from a body fluid of a subject having such tumor, and the use of an interaction partner of a high mobility group protein of the HMGA family in this kind of methods.

A big challenge in prenatal diagnosis is to detect fetal cells or preferably cell-free DNA, respectively, in maternal blood or urine thus allowing to perform non-invasive prenatal diagnosis. Also a big challenge is to select and isolate tumor DNA in body fluid of cancer patients.

Often, these attempts are difficult to perform due to the absence of tools that allow to detect specifically cells of fetal origin. E.g. the detection of circulating trophoblast cells in maternal blood is challenging due to a lack of a reproducible trophoblast-specific antibody (Tjoa et al., 2007). On the other hand, the availability of these tools would greatly improve the methods to prenatal diagnosis of fetal chromosomal abnormalities because once detected fetal cells can easily be used for analyzing their chromosomal complement by using molecular cytogenetic methods as in particular fluorescence in situ hybridization (FISH).

The problem underlying the present invention is thus to provide a means which allow the detection of fetal and embryonic cells in maternal blood and body fluid.

A further problem underlying the present invention is to provide a means for the separation of fetal and embryonic cells from a maternal blood and body fluid.

A still further problem underlying the present invention is to provide a means for the separation of fetal and embryonic chromatin and/or fetal and embryonic preferably cell-free nucleic acids in a maternal body fluid including blood.

Another problem underlying the present invention is to provide a means for distinguishing fetal and embryonic cells from maternal cells and/or maternal tissue and for distinguishing fetal and embryonic cell-free DNA from maternal cell-free DNA.

A further problem underlying the present invention is to provide a means for the detection and/or preferably selective isolation of cell-free chromatin and/or a nucleic acids from a tumor, preferably from tumor cells and for distinguishing chromatin and/or nucleic acids from the tumor from those derived from normal cells.

These and other problems underlying the present invention are solved by the subject matter of the attached independent claims. Preferred embodiments may be taken from the dependent claims.

In a first aspect the problem underlying the present invention is solved by a method for the detection of fetal and embryonic cells in a maternal body fluid comprising the steps of
a) providing a sample of a maternal body fluid containing or presumed to contain fetal and/or embryonic cells,
b) providing an interaction partner of a high mobility group protein of the HMGA family;
c) reacting the sample with the interaction partner, whereupon preferably a complex is formed between the interaction partner and the fetal and embryonic cells; and
d) detecting the complex.

In a second aspect the problem underlying the present invention is solved by a method for the separation and/or enrichment of fetal and embryonic cells contained in a maternal body fluid comprising the steps of
a) providing a sample of the maternal body fluid,
b) providing an interaction partner of a high mobility group protein of the HMGA family;
c) reacting the sample with the interaction partner, whereupon preferably a complex is formed between the interaction partner and the fetal and embryonic cells; and
d) separating the complex from the sample.

In an embodiment of the second aspect the separating is made through precipitation or chromatography.

In a third aspect aspect the problem underlying the present invention is solved by a method for the detection of fetal and/or embryonic chromatin and/or fetal and/or embryonic nucleic acid(s) in a maternal body fluid comprising the steps of
a) providing a sample of a maternal body fluid containing or presumed to contain fetal and/or embryonic chromatin and/or fetal and/or embryonic nucleic acid(s),
b) providing an interaction partner of a high mobility group protein of the HMGA family;
c) reacting the sample with the interaction partner, whereupon preferably a complex is formed between the interaction partner and the fetal and/or embryonic chromatin and/or fetal and/or embryonic nucleic acid(s); and
d) detecting the complex.

In a fourth aspect the problem underlying the present invention is solved by a method for the separation and/or enrichment of fetal and embryonic cells contained in a maternal body fluid comprising the steps of
a) providing a sample of a maternal body fluid,
b) providing an interaction partner of a high mobility group protein of the HMGA family;
c) reacting the sample with the interaction partner, whereupon preferably a complex is formed between the interaction partner and fetal and/or embryonic chromatin and/or fetal and/or embryonic nucleic acid(s); and
d) separating the complex from the sample.

In an embodiment of the fourth aspect the separating is made through precipitation or chromatography.

In a fifth aspect the problem underlying the present invention is solved by a method the detection of fetal and/or embryonic cells in a maternal body fluid comprising the steps of
a) providing a sample of a maternal body fluid containing or presumed to contain fetal and/or embryonic cells,
b) providing a primer or a probe specific for a gene or transcript of such gene encoding a high mobility group protein of the HMGA family;
c) reacting the sample with the primer or probe, whereupon the primer or probe binds to the gene or transcript contained in the fetal and embryonic cells; and
d) detecting the hybridization or the transcript(s), preferably obtained by the use of the primer upon running a polymerase chain reaction.

In an embodiment of the fifth aspect the primer or the probe is introduced into the fetal and/or embryonic cells.

In a sixth aspect the problem underlying the present invention is solved by a method the detection of fetal and/or embryonic chromatin and/or fetal and/or embryonic nucleic acid(s) in a maternal body fluid comprising the steps of
a) providing a sample of a maternal body fluid containing or presumed to contain fetal and/or embryonic chromatin and/or fetal and/or embryonic nucleic acid(s),
b) providing a primer or a probe specific for a gene or transcript of such gene encoding a high mobility group protein of the HMGA family;
c) reacting the sample with the primer or probe, whereupon the primer or probe binds to the gene or transcript contained in the fetal and embryonic cells; and
d) detecting the hybridization or the transcript(s), preferably obtained by the use of the primer upon running a polymerase chain reaction, preferably a quantitative polymerase chain reaction.

In an embodiment of any of the first to the sixth aspect the maternal body fluid is selected from the group comprising urine, blood and transcervical lavage.

In a seventh aspect the problem underlying the present invention is solved by a method for distinguishing fetal and/or embryonic cells from maternal cells and/or maternal tissue comprising the following steps
a) providing a sample containing both fetal and/or embryonic cells, and maternal cells and/or maternal tissue;
b) providing an interaction partner of a high mobility group protein of the HMGA family;
c) reacting the sample with the interaction partner, whereupon preferably a complex is formed between the interaction partner and the high mobility group protein of the HMGA family or the gene or transcript coding therefore;
d) with the embryonic cells or fetal cells exhibiting an more of the complex compared to the maternal tissue and/or maternal cells.

In an embodiment of the seventh aspect the maternal cells and/or maternal tissue is selected from the group comprising maternal placenta tissue and placental stromal cells.

In an embodiment of the seventh aspect the fetal and embryonic cells are from the trophoblast and the cytotrophoblast.

In an embodiment of the seventh aspect the fetal and embryonic cells and the maternal cells and/or the maternal tissue is contained in a sample obtained by chorionic villi sampling.

In an eighth aspect the problem underlying the present invention is solved by a method for the detection of chromatin and/or nucleic acid(s) from a tumor or cancer of a subject having the tumor or cancer comprising the following steps:
a) providing a sample of a body fluid of the subject having the tumor or cancer or suspected of having the tumor or cancer;
b) providing an interaction partner of a high mobility group protein of the HMGA family;
c) reacting the sample with the interaction partner whereupon preferably a complex is formed between the interaction partner and the high mobility group protein of the HMGA family;
d) detecting the complex.

In a ninth aspect the problem underlying the present invention is solved by a method for the separation, isolation and/or enrichment of chromatin and/or nucleic acid(s) from a tumor or cancer of a subject having the tumor comprising the following steps:
a) providing a sample of a body fluid of the subject having the tumor or cancer or suspected of having the tumor or cancer;
b) providing an interaction partner of a high mobility group protein of the HMGA family;
c) reacting the sample with the interaction partner whereupon preferably a complex is formed between the interaction partner and the high mobility group protein of the HMGA family;
d) separating the complex from the sample.

In an embodiment of the ninth aspect 17 the separating is made through precipitation or chromatography.

In an embodiment of the eighth and ninth aspect the body fluid has had contact with the cells forming the tumor.

In an embodiment of the eighth and ninth aspect the sample contains cells from the tumor or cancer.

In an embodiment of the eighth and ninth aspect the body fluid is selected from the group comprising blood, urine, sputum, effusions, lavage, stool and saliva.

In an embodiment of the eighth and ninth aspect the body fluid is selected from the group comprising saliva, stool and blood.

In an embodiment of the eighth and ninth aspect the chromatin and/or the nucleic acid is contained in a sample from a subject assumed to suffer from or being at risk to develop a tumor or cancer, whereby the sample is preferably selected from the group comprising urine, blood, serum, transcervical lavage, sputum, pleural an effusions, ascitic effusions, saliva, biopsies and stool.

In an embodiment of the eighth and ninth aspect the tumor or cancer is selected from the group comprising malignant epithelial cancers, malignant mesenchymal tumors, tumors of endocrine and neuroendocrine origin, leukemias, and lymphomas.

In an embodiment of the eighth and ninth aspect the tumor or cancer is selected from the group comprising lung cancer, breast cancer, non-small cell lung cancer, colorectal cancer, ovarian cancer, endometrial cancer, prostate cancer, and pancreatic cancer.

In an embodiment of any of the first to the ninth aspect the nucleic acid is selected from the group comprising DNA, mRNA, pre-mRNA and processed mRNA.

In an embodiment of any of the first to the ninth aspect the nucleic acid is a nucleic acid which binds to a high mobility group protein of the HMGA family, preferably HMGA1 and/or HMGA2.

In an embodiment of any of the first to the ninth aspect the interaction partner is selected from the group comprising antibodies, peptide aptamers, anticalines, aptamers, spiegelmers, promers and probes to the high mobility group protein of the HMGA family or the gene or transcript coding therefore.

In a tenth aspect the problem underlying the present invention is solved by the use of an interaction partner of a high mobility group protein of the HMGA family for the detection of fetal and embryonic cells in a maternal body fluid.

In an eleventh aspect the problem underlying the present invention is solved by the use of an interaction partner of a high mobility group protein of the HMGA family for the separation of fetal and embryonic cells from a maternal body fluid.

In an embodiment of the tenth and eleventh aspect the high mobility group protein of the HMGA family is selected from the group comprising HMGA1 and HMGA2.

In an embodiment of the tenth and eleventh aspect the interaction partner is selected from the group comprising antibodies, peptide aptamers, anticalines, aptamers and spiegelmers.

In an embodiment of the tenth and eleventh aspect the maternal body fluid is selected from the group comprising urine, blood, and transcervical lavage.

In an 12^{th} aspect the problem underlying the present invention is solved by the use of an interaction partner of a high mobility group protein of the HMGA family for the detection of fetal and embryonic chromatin and/or fetal and embryonic nucleic acids in a maternal body fluid.

In an 13^{th} aspect the problem underlying the present invention is solved by the use of an interaction partner of a high mobility group protein of the HMGA family for the separation of fetal and embryonic chromatin and/or fetal and embryonic nucleic acids in a maternal body fluid.

In an embodiment of the 12^{th} and 13^{th} aspect the high mobility group protein of the HMGA family is selected from the group comprising HMGA1 and HMGA2.

In an embodiment of the 12^{th} and 13^{th} aspect the interaction partner is selected from the group comprising antibodies, peptide aptamers, anticalines, aptamers and spiegelmers.

In an embodiment of the 12^{th} and 13^{th} aspect the maternal body fluid is selected from the group comprising urine, blood, and transcervical lavage.

In an embodiment of the 12^{th} and 13^{th} aspect the nucleic acid is selected from the group comprising DNA, mRNA, pre-mRNA and processed mRNA.

In an embodiment of the 12^{th} and 13^{th} aspect the nucleic acid is a nucleic acid which binds to a high mobility group protein of the HMGA family, preferably HMGA1 and/or HMGA2.

In an 14^{th} aspect the problem underlying the present invention is solved by the use of an interaction partner of a high mobility group protein of the HMGA family for distinguishing fetal and embryonic cells from maternal cells and/or maternal tissue.

In a 15^{th} aspect the problem underlying the present invention is solved by the use the maternal cells and/or maternal tissue is selected from the group comprising maternal placenta tissue and placental stromal cells.

In an embodiment of the 14^{th} and 15^{th} aspect the fetal and embryonic cells are from the trophoblast and the cytotrophoblast.

In an embodiment of the 14^{th} and 15^{th} aspect the fetal and embryonic cells and the maternal cells and/or the maternal tissue is contained in a sample obtained by chorionic villi sampling.

In an embodiment of the 14^{th} and 15^{th} aspect the high mobility group protein of the HMGA family is selected from the group comprising HMGA1 and HMGA2.

In an embodiment of the 14^{th} and 15^{th} aspect the interaction partner is selected from the group comprising antibodies, peptide aptamers, anticalines, aptamers and spiegelmers.

In a 16^{th} aspect the problem underlying the present invention is solved by the use of a polymerase chain reaction for the amplification of a nucleic acid coding for a high mobility group protein of the HMGA family in a method for the detection of fetal and embryonic cells in maternal body fluids.

In an embodiment of the 16^{th} aspect the polymerase chain reaction is a RT-PCR.

In a 17^{th} aspect the problem underlying the present invention is solved by the use of a probe specifically interacting and/or hybridising with a nucleic acid coding for a high mobility group protein of the HMGA family for the detection and/or amplification of fetal and embryonic cells in maternal body fluids.

In a 18^{th} aspect the problem underlying the present invention is solved by the use of a primer specifically interacting and/or hybridising with a nucleic acid coding for a high mobility group protein of the HMGA family for the detection and/or amplification of fetal and embryonic cells in maternal body fluids.

In an embodiment of the 16^{th} to the 18^{th} aspect the polymerase chain reaction is a quantitative polymerase chain reaction.

In an embodiment of the 16^{th} to the 18^{th} aspect the high mobility group protein of the HMGA family is selected from the group comprising HMGA1 and HMGA2.

In an embodiment of the 16^{th} to the 18^{th} aspect the maternal body fluid is selected from the group comprising urine, blood, and transcervical lavage.

In a 19^{th} aspect the problem underlying the present invention is solved by the use of an interaction partner of a high mobility group protein of the HMGA family for the detection and/or isolation of chromatin and/or a nucleic acids from a tumor, preferably tumor cells, whereby the tumor cells are circulating in the blood of a subject, in a sample of blood of a subject or present in a body fluid of a subject or a sample of a body fluid of a subject, whereby the body fluid is preferably selected from the group comprising sputum, urine, effusions, lavage, or whereby the sample is a sample known to may have had contact with cancer cells, whereby the sample is preferably selected from the group comprising stool and saliva.

In an embodiment of the 19^{th} aspect the interaction partner is selected from the group comprising antibodies, peptide aptamers, anticalines, aptamers and spiegelmers.

In an embodiment of the 19^{th} aspect the high mobility group protein of the HMGA family is selected from the group comprising HMGA1 and HMGA2.

In an embodiment of the 19^{th} aspect the chromatin and/or the nucleic acid is contained in a sample from a subject assumed to suffer from or being at risk to develop a tumor, whereby the sample is preferably selected from the group comprising urine, blood, serum, transcervical lavage, sputum, pleural an effusions, ascitic effusions, saliva, biopsies and stool.

In an embodiment of the 19^{th} aspect the nucleic acid is selected from the group comprising DNA, mRNA, pre-mRNA and processed mRNA.

In an embodiment of the 19^{th} aspect the nucleic acid is a nucleic acid which binds to a high mobility group protein of the HMGA family, preferably HMGA1 and/or HMGA2.

In an embodiment of the 19^{th} aspect the tumor is selected from the group comprising malignant epithelial cancers, malignant mesenchymal tumors, tumors of endocrine and neuroendocrine origin, leukemias, and lymphomas.

In an embodiment of the 19^{th} aspect the tumor is selected from the group comprising lung cancer, breast cancer, non-small cell lung cancer, colorectal cancer, ovarian cancer, endometrial cancer, prostate cancer, and pancreatic cancer.

It will be understood by a person skilled in the art that the methods of the present invention are preferably in vitro methods.

In an embodiments, the fetal cells, embryonic cells and tumor cells may be lysed prior or subsequently to the reacting of the sample and the interaction partner.

In a preferred embodiment, the complex to which it is referred in the various methods according to the present invention is one which consists of the interaction partner, a high mobility group protein of the HMGA family and one or several nucleic acid molecules which are preferably bound to or by said high mobility group protein of the HMGA family.

The present inventor has surprisingly found that a high mobility group protein of the HMGA family is not only strongly expressed by fetal and embryonic cells but also binds to cell-free DNA released e.g. from fetal or embryonic cells or from tumor cells. Such strong expression thus allows for the detection of fetal and embryonic cell and separation, particularly in view of a background of other tissues and cells, preferably maternal cells and tissues, respectively. Additionally, such strong expression allows the separation of fetal and embryonic cells and issues from non-fetal and non-embryonic cells and tissues, preferably from maternal cells and tissues. By detecting and separating embryonic and fetal cells based on said strong expression, also embryonic and fetal preferably cell-free nucleic acid is made available which may be detected and separated.

The present inventor has furthermore surprisingly found that embryonic and fetal nucleic acid may also be isolated and/or separated from maternal blood and/or body fluid based on the finding that embryonic and fetal chromatin and nucleic acid is present as complex with a high mobility group protein of the HMGA family and more specifically with HMGA2.

In both cases, the embryonic and fetal nucleic acid and chromatin, respectively, may then be subject to an analysis, for example for prenatal diagnosis. There are methods known in the art which will allow a person skilled in the art to further characterize the nucleic acid and chromatin, respectively, thus detected and, optionally, separated.

Still further, the present inventor has found that a sample from a tumor patient contains a high mobility protein of the HMGA family and may be used as an indicator or marker for said tumor. More specifically, such protein, i.e. the protein of the HMGA family, is available as a complex with one or several nucleic acid molecules or with chromatin which are/is derived from the tumor. It is based on such nucleic acid molecules and chromatin, respectively, that the tumor can be diagnosed and further characterised. Without wishing to be bound by any theory, the present inventor understands that the nucleic acid molecule and chromatin provides for a finger-print of the individual tumor entity, whereby the nucleic acid and chromatin of such tumor entity, respectively, interact with the HMGA protein due to the general binding characteristics of high mobility proteins with nucleic acids as is generally known in the art. There are methods known in the art which will allow a person skilled in the art to further characterize the nucleic acid and chromatin, respectively, thus detected and, optionally, separated.

Without wishing to be bound by any theory, the present inventor assumes that particularly those tumors and cancers may be subject to the various method of the instant application and invention, respectively, which show an invasive growth and/or are associated with neoangionesis which explains the presence of the complexes in the body fluids and in particular in the blood and lymph fluid. However, it is to be acknowledged that the instant invention is not limited to this type of tumors.

It is within the present invention that the detection of the high mobility group protein of the HMGA family is made using an interaction partner of said high mobility group protein. Such interaction partner is preferably selected from the group comprising antibodies, peptide aptamers, anticalines, aptamers and spiegelmers which are as such known in the art and can be generated by a person skilled in the art based on her/his respective knowledge, particularly also in view of the teaching of the present application. In connection with this kind of interaction partner, it has to be acknowleged that a high mobility group protein of the HMGA family as defined herein or a part thereof is the target. It is also within the present invention that the target is a structure or epitope which is generated upon the interaction of the high mobility group protein of the HMGA family and the nucleic acid and/or chromatin of the fetus, embryo and tumor, respectively.

The manufacture of an antibody specific for the target is known to the person skilled in the art and, for example, described in Harlow, E., and Lane, D., “Antibodies: A Laboratory Manual,” Cold Spring Harbor Laboratory, Cold Spring Harbor, N.Y., (1988). Preferably, monoclonal antibodies may be used in connection with the present invention, which may be manufactured according to the protocol of Köhler and Milstein and further developments based thereon. Antibodies as used herein, include, but are not limited to, complete antibodies, antibody fragments or derivatives such as Fab fragments, Fc fragments and single-stranded antibodies, or anticalins, as long as they are suitable and capable of binding to the target. Apart from monoclonal antibodies also polyclonal antibodies may be used and/or generated. The generation of polyclonal antibodies is also known to the one skilled in the art and, for example, described in Harlow, E., and Lane, D., “Antibodies: A Laboratory Manual,” Cold Spring Harbor Laboratory, Cold Spring Harbor, N.Y., (1988).

The antibodies, which may be used according to the present invention, may have one or several markers or labels. Such markers or labels may be useful for detecting the antibody either in its diagnostic application or its therapeutic application. Preferably the markers and labels are selected from the group comprising avidin, streptavidin, biotin, gold and fluorescein and used, e.g., in ELISA methods. These and further markers as well as methods are, e.g. described in Harlow, E., and Lane, D., “Antibodies: A Laboratory Manual,” Cold Spring Harbor Laboratory, Cold Spring Harbor, N.Y., (1988). Additionally or alternatively, the antibodies as well as any other target antagonist or interaction partner described herein may be a labelled antagonist as more generally described herein.

It is also within the present invention that the label or marker exhibits an additional function apart from detection, such as interaction with other molecules. Such interaction may be, e.g., specific interaction with other compounds. These other compounds may either be those inherent to the system where the antibody is used such as the human or animal body or to the sample which is analysed by using the respective antibody. Appropriate markers may, for example, be biotin or fluoresceine with the specific interaction partners thereof such as avidin and streptavidin and the like being present on the respective compound or structure to interact with the thus marked or labelled antibody. Again this applies also to the other target interaction partners described herein such as aptamers and spiegelmers.

Another class of interaction partners are anticalines which are, among others, described in German patent application DE 197 42 706.

A further class of interaction partners which can be used in a way identical to the antibodies, and thus for the same purposes, are the so-called peptide-aptamers. Using a target, peptide aptamers can be generated using a screening process making use of a polypeptide library as described herein in more detail. The selection criterion is that the selected polypeptide is actually and specifically binding to the target.

More specifically, such peptide aptamers may be generated by using methods according to the state of the art such as phage display. Basically, a library of peptides is generated, such as in the form of phages, and this kind of library is contacted with the target molecule. Those peptides binding to the target molecule are subsequently removed from the respective reaction, preferably as a complex with the target molecule. It is known to the one skilled in the art that the binding characteristics, at least to a certain extent, depend on the particularly realized experimental set-up such as salt concentration and the like. After separating those polypeptides binding to the target molecule with a higher affinity or a bigger force, from the non-binding members of the library, and optionally also after removal of the target molecule from the complex of target molecule and polypeptide, the respective polypeptide(s) may subsequently be characterised. Prior to the characterisation optionally an amplification step is realized such as, e.g., by propagating the polypeptide coding phages. The characterisation preferably comprises the sequencing of the target binding polypeptides and ultimately of those polypeptides acting as antagonists or interaction partners of the target as defined herein. Basically, the polypeptides are not limited in their length, however, preferably polypeptides having a length from about 8 to 20 amino acids are preferably obtained in the respective methods. The size of the libraries may be about 10² to 10¹⁸, preferably 10⁸ to 10¹⁵ different polypeptides, however, is not limited thereto.

A further class of interaction partners which may be used in accordance with the present invention are aptamers. Aptamers are D-nucleic acids which are either single stranded or double stranded and which specifically interact with a target molecule. The manufacture or selection of aptamers is, e.g., described in European patent EP 0 533 838. Basically the following steps are realized. First, a mixture of nucleic acids, i.e. potential aptamers, is provided whereby each nucleic acid typically comprises a segment of several, preferably at least eight subsequent randomised nucleotides. This mixture is subsequently contacted with the target molecule, whereby the nucleic acid(s) bind to the target molecule, such as based on an increased affinity towards the target or with a bigger force thereto, compared to the candidate mixture. The binding nucleic acid(s) are/is subsequently separated from the remainder of the mixture. Optionally, the thus obtained nucleic acid(s) is amplified using, e.g. polymerase chain reaction. These steps may be repeated several times giving at the end a mixture of nucleic acids having an increased ratio of nucleic acids specifically binding to the target from which the final binding nucleic acid is then optionally selected. These specifically binding nucleic acid(s) are referred to as aptamers. It is obvious that at any stage of the method for the generation or identification of the aptamers samples of the mixture of individual nucleic acids may be taken to determine the sequence thereof using standard techniques. It is within the present invention that the aptamers may be stabilized such as, e.g., by introducing defined chemical groups which are known to the one skilled in the art of generating aptamers. Such modification may for example reside in the introduction of an amino group at the 2′-position of the sugar moiety of the nucleotides. Aptamers are currently used as both therapeutic and diagnostic agents. However, it is also within the present invention that the thus selected or generated aptamers are suitable in the practicing of the various method according to the present invention.

A still further class of interaction partners which may be used in connection with the present invention are spiegelmers. Spiegelmers are a special form of aptamers. The generation or manufacture of spiegelmers which may be used or generated according to the present invention using the target is based on a similar principle. The manufacture of Spiegelmers is described in international patent application WO 98/08856. Spiegelmers are L-nucleic acids, which means that they are composed of L-nucleotides rather than aptamers which are composed of D-nucleotides. Spiegelmers are characterized by the fact that they have a very high stability in biological system and, comparable to aptamers, specifically interact with the target molecule against which they are directed. In the purpose of generating Spiegelmers, a heterogenous population of D-nucleic acids is created and this population is contacted with the optical antipode of the target molecule, in the present case for example with the D-enantiomer of the naturally occurring L-enantiomer of the target. Subsequently, those D-nucleic acids are separated which do not interact with the optical antipode of the target molecule. However, those D-nucleic acids interacting with the optical antipode of the target molecule are separated, optionally determined and/or sequenced and subsequently the corresponding L-nucleic acids are synthesized based on the nucleic acid sequence information obtained from the D-nucleic acids. These L-nucleic acids which are identical in terms of sequence with the aforementioned D-nucleic acids interacting with the optical antipode of the target molecule, will specifically interact with the naturally occurring target molecule rather than with the optical antipode thereof. Similar to the method for the generation of aptamers it is also possible to repeat the various steps several times and thus to enrich those nucleic acids specifically interacting with the optical antipode of the target molecule.

It will also be acknowledged by a person skilled in the art that also primers and probes may be used as some sort of interaction partner whereby these interaction partners, of course, targeting the nucleic acid coding for a/the high mobility group protein of the HMGA family. The design of the such primer and probe, respectively, is within the skills of a person of the art.

The present invention shall now be further illustrated by way of example using an antibody against a high mobility group protein of the HMGA family.

The present application describes results of a study using the expression of high mobility group proteins of the HMGA family to select and characterize fetal and embryonic cells present in the maternal blood as well as in maternal urine. By both an antibody against HMGA2 as well as by quantitative RT-PCR for the mRNA of its gene the present inventor was able to show that fetal cells in maternal blood and urine are characterized by high levels of HMGA2. Accordingly, the transcription product of the gene as well as its translation product can be used for the selection of fetal and embryonic cells.

Cells from a urine sample are separated by appropriate methods such as e.g. centrifugation and then transferred to a slide. By conventional well-established immunocytochemistry (ICC) antibodies against HMGA2 can then be used to stain cells that are positive for this protein and accordingly are of embryonic or fetal origin. The antibodies used are taken from the group of monoclonal or polyclonal antibodies. Subsequently, only those cells positive for HMGA2 can be subjected to further cytogenetic, molecular cytogenetic and molecular analyses as e.g. FISH or PCR.

Alternatively, the selection of embryonic cells or fetal cells can also be performed successfully by cell sorting, preferably fluorescence activated cell sorting, of those cells that stained positive with the anti-HMGA2 antibody. Preferably the interaction partner provides for a respective label which allows the application of this kind of technique.

In another aspect of the present invention antibodies against HMGA proteins are used for immunoprecipitation of chromatin complexes containing HMGA proteins and DNA. Immunoprecipitation of chromatin is a well suited method to identify specific protein-DNA interaction e.g. to study interactions between transcription factors and DNA and, e.g. described by Dahl J. A (Dahl J A, Collas P. A quick and quantitative chromatin immunoprecipitation assay for small cell samples. Front Biosci. 2007 Sep. 1; 12:4925-31) or Haring M. et al. (Haring M, Offermann S, Danker T, Horst I, Peterhaensel C, Stam M. Chromatin immunoprecipitation: optimization, quantitative analysis and data normalization.

It is well known that blood, body fluids, sputum, stool, ammiotic fluids contain cell-free DNA which can be used for further analyses (Hsu et al, 2007, Levenson, 2007, Schwarzenbach et al, 2007, Zanetti-Dällenbach et al, 2007). However, it is a so far unsolved problem to isolate a specific DNA from these sources or e.g. embryonic or fetal DNA from maternal blood (Maron et al, 2007, Tsang and Lo, 2007) (fetal/embryonic vs. maternal DNA) or to isolate specifically tumor DNA from e.g. sputum, urine, or blood (DNA from tumor cells vs. DNA from normal cells).

The present invention aims at the specific isolation of DNA molecules associated with HMGA, and in particular HMGA2 proteins i.e. those DNAs of fetal/embryonic or neoplastic cells.

Herein, the invention relates to the isolation and enrichment, respectively, of those DNA fragments that are associated with any type of HMGA protein but in particular with HMGA2. HMGA2 and the other HMGA proteins are known to be abundantly expressed in cancer cells and fetal and embryonic cells. In our experiments the association between HMGA proteins and their DNA binding partner was found to be highly stable. Thus, their complexes can also be found and isolated from other samples than tissues and cells. In case of cancer they e.g. circulate in the blood, can be isolated from sputum and stool or from malignant effusions. Thus, their complexes can be used to specifically pick up DNA fragments that are derived from cancer cells from all these materials that have had contact with cancer cells, including, but not limited to stool and saliva. This is an obvious breakthrough for screening of early i.e. small cancers from a variety of samples because in almost every cancer entity dead cells do appear at relatively early stages then releasing their chromatin fragments. Certainly, basically the same method can also be used to isolate fetal chromatin from maternal blood and represents a major step forward in a non-invasive prenatal diagnosis.

It will be acknowledged that the term high mobility group protein of the HMGA family also comprises fragments of such a protein. Preferably, such protein has the characteristics of the whole protein or at least one of its AT-hooks. As used herein, the term high mobility group protein HMGA1 includes two variants, i.e. HMGA1a and HMGA1b.

Antibodies against HMGA proteins, in particular HMGA2 and other molecular binding specifically to HMGA proteins and HMGA2, and as aptameres, spiegelmers, peptide aptamers and anticalines can be used to specifically isolate tumor DNA. These molecules can either target the whole protein or particular domains of it. Off particular interest as target domains are those parts of the protein that bind to the DNA i.e. the so-called AT-hooks as described in the art (Wisniewski and Schwanbeck, 2000). In any case, the molecules incl. the antibodies that specifically target the HMGA proteins are the tool to isolate HMGA-tumor DNA complexes and then to remove the DNA from the complex.

There are several well established techniques to isolate the protein-DNA complex as e.g. chromatin immunoprecipitation (Dahl and Collas, 2007, Gao et al, 2007, Haring et al, 2007, McCann et al, 2007), binding the antibody or alternative specifically binding molecules to a matrix etc. which can be used to meet the challenges related to that invention. Prior to isolation the HMGA protein can be covalently linked to its target DNA by well-established methods e.g. cross-linking by formalin but due to both the high affinity of HMGA proteins to DNA and the high stability of the complex this step can be deleted as well. In the practising of the invention also methods for the stabilization of the complex consisting of DNA and chromatin and the complex formed by the addition of the interaction partner to such complex (Kuras L. 2004. Characterization of protein-DNA association in vivo by chromatin immunoprecipitation. Methods Mol Biol.; 284:147-62).

After appropriate isolation of the complexes well-established methods exists to remove the DNA from the complex. This kind of procedure is further illustrated in the example part and more specifically referred to there as decross-linking.

These DNA molecules, respectively, can than be analysed for changes that allow to detect a cancer (Helmig and Schneider, 2007, Taback et al, 2006) or are associated with a particular prognostic subgroup of a cancer disease (Chin et al, 2007) or are associated with a particular subgroup of a cancer that may allow to predict the success of any type of therapy (Riesterer et al, 2007).

Among the methods to characterize the DNA and the changes that characterize either tumor DNA or specific entities or subgroups as outlined above are:
Specific/non random methylation patterns of the DNA (Jing et al, 2007, Ongenaert et al, 2007)
The presence of either type of mutation recognisable e.g. by DNA sequencing (Soussi and Wiman, 2007), loss of heterozygosity (Schwarzenbach et al., 2007), restriction enzyme digestion, solid phase oligonucleotide ligation assay, PCR, qPCR, mass-spectometry (Hwang and Bowen, 2007), SNP analysis
Genomic inbalances that can be detected by CGH and matrix-CGH (Climent, et al, 2007) i.e. hybridization of said DNA to Chips (van Beers and Nederlof, 2007) or chromosomes (Dehan et al, 2007), mass-spectometry (Ollikainen et al, 2007), . . . .
Microsatelite analyses (Castagnaro et al, 2007)

Methods for prenatal diagnosis are known to a person skilled in the art and are reported for example in the journal “Prenatal Diagnosis”.

The invention is now further illustrated by reference to the figures and examples from which further features, embodiments and advantages may be taken.

FIG. 1 shows the nucleic acid sequence coding for HMGA2;

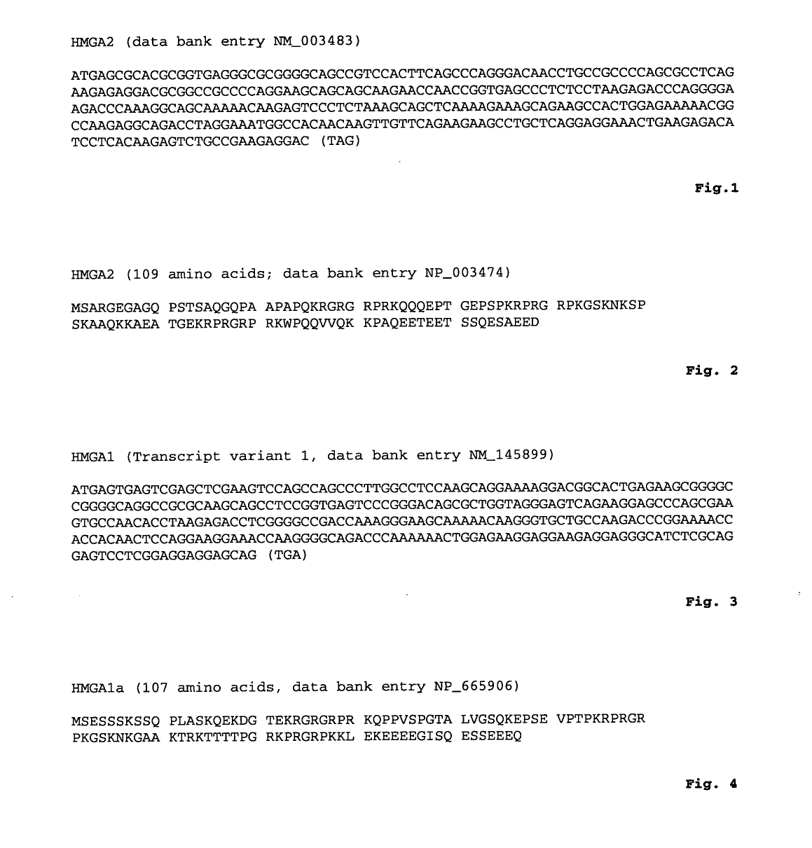

FIG. 2 shows the amino acid sequence of HMGA2;

FIG. 3 shows the amino acid sequence coding for HMGA1, more specifically variant 1;

FIG. 4 shows the amino acid sequence of HMGA1a;

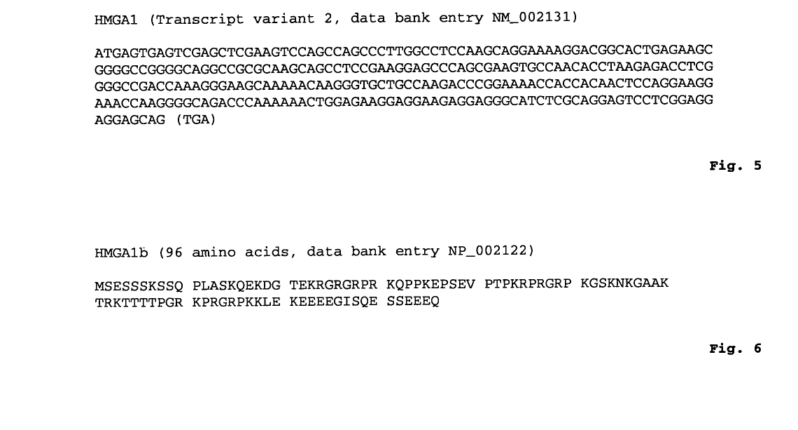

FIG. 5 shows the nucleic acid sequence coding for HMGA1, more specifically variant 2;

FIG. 6 shows the amino acid sequence of HMGA1b;

### EXAMPLE 1

### Use of HMGA Based Chromatin Selection (HACS) for the Selection of Fetal DNA from Maternal Serum

Serum samples from eight pregnant women ranging between the 14^{th} and 16^{th} week of gestation have been used for the HACS technology followed by quantitative PCR. All women had asked for prenatal cytogenetic diagnosis after amniocentesis. 5 ml serum have been used for chromatin immunoprecipitation using an antibody against HMGA2 according to routine protocols for chromatin immunoprecipitation. Briefly, formalin has been added for cross-linking to preserve the chromatin structure for isolation and the immunoprecipitation procedure. Chromatin immunoprecipitation has then been performed using a polyclonal antibody against HMGA2 (e.g. Stanta Cruz Biotechnology Inc, Santa Cruz, USA) and decross-linking of the DNA is performed. Decross-linked DNA is then used to quantitative PCR experiments. For these two primer pairs along with appropriate probes for real time PCR (Taqman, Applied Biosystems, Foster City, USA) of the Y chromosome and chromosome 21 were used. In three of the eight samples Y-specific sequences could be detected and the ratio for Y chromosome sequences versus chromosome 21 specific sequences was 0.41:1, 0.39:1, and 0.33:1, respectively. After amniocentesis and prenatal chromosome diagnosis, for these women a fetus with an apparently normal male karyotype was diagnosed. In the remaining five cases, a female karyotype was found. This indicates, that preferentially fetal DNA was selected.

### EXAMPLE 2

### Use of HMGA Based Chromatin Selection (HACS) for the Selection of Tumor DNA from Patient's Serum

A serum sample from a patient suffering from lung cancer (adenocarcinoma) was taken before initial surgery. There was no evidence for distant metastases. 5 ml serum have been used for chromatin immunoprecipitation using an antibody against HMGA2 according to routine protocols for chromatin immunoprecipitation. Briefly, formalin has been added for cross-linking to preserve the chromatin structure for isolation and the immunoprecipitation procedure. Chromatin immunoprecipitation has then been performed using a polyclonal antibody against HMGA2 (Santa Cruz Biotechnology Inc., Santa Cruz, USA) and decross-linking of the DNA is performed. Decross-linked DNA is then used to quantitative PCR experiments. For these experiments two primer pairs along with appropriate probes for real time PCR (Taqman, Applied Biosystems, Foster City, USA) of chromosome 21 and chromosome 8 were used. In the sample both chromosome 8- and 21-specific sequences could be detected and the ratio for 8 chromosome sequences versus chromosome 21 specific sequences was 2.9:1. The patient had an apparently normal 46,XY karyotype. This indicates that preferentially tumor DNA was selected and that the tumor had a polysomy of chromosome 8.

### EXAMPLE 3

### Existence of Numerous and Well-Spread Consensus Recognition DNA Sequences for HMGA2 in the Human Genome

Quite recently, consensus sequences for high affinity binding of HMGA2 to its target DNA have been described (Cui and Leng, Specific recognition of AT-rich DNA sequences by the Mammalian High Mobility Group Protein AT-hook 2: A SELEX Study, Biochemistry, published on Web Oct. 23, 2007). We have used these sequences for an in silico analysis of their distribution among the human genome. As shown in table 1 there is a great number of these consensus sequences spread over the human chromosomes.

| | | Chromosome | Targets |
|---|---|---|---|
| | Targets per | length | per base |
| Chromosome | chromosome | (bp) | pairs |
| 1 | 2104 | 226212984 | 107515 |
| 2 | 2402 | 237898220 | 99041 |
| 3 | 1984 | 195304083 | 98439 |
| 4 | 2094 | 187939711 | 89751 |
| 5 | 1871 | 177846453 | 95054 |
| 6 | 1704 | 169099554 | 99236 |
| 7 | 1520 | 155402083 | 102238 |
| 8 | 1431 | 143330736 | 100161 |
| 9 | 1123 | 120989686 | 107737 |
| 10 | 1185 | 131738012 | 111171 |
| 11 | 1195 | 131246147 | 109829 |
| 12 | 1211 | 130303534 | 107599 |
| 13 | 1037 | 95746838 | 92330 |
| 14 | 817 | 88290585 | 108066 |
| 15 | 723 | 81926261 | 113314 |
| 16 | 611 | 78990239 | 129280 |
| 17 | 551 | 79617833 | 144496 |
| 18 | 772 | 74660417 | 96710 |
| 19 | 277 | 56037509 | 202301 |
| 20 | 426 | 59505253 | 139683 |
| 21 | 334 | 35451691 | 106142 |
| 22 | 220 | 35058650 | 159357 |
| X | 1627 | 152577922 | 93778 |
| Y | 236 | 25652954 | 108698 |

In general, a high affinity binding sequence occurs on average roughly every 90,000-100,000 base pairs. Thus, knowledge of these sequences and their distribution can be helpful in further refining the methods for the use of chromatin selected by HACS as e.g. quantitative PCR.

### EXAMPLE 4

### Detailed Operating Procedure

The following is a detailed operating procedure which describes in more detail the preparation of embryonic DNA from amniotic fluid.

Amniotic fluid is centrifuged at 100×g for 10 min, and 2 ml of the supernatant are transferred to a 13 ml tube. To cross-link the protein DNA complexes, 37% formaldehyde is added to a final concentration of 1%. After 10 min of incubation at room temperature, the cross-linking reaction is stopped by adding 1 M glycine to a final concentration of 0.125 mM. 10% of the sample serve as input control and are transferred to a 1.5 ml tube and frozen at −20° C. The remaining sample is divided by half and transferred to two 1.5 ml tubes, and 50 μl of Protein A/G PLUS-Agarose are added to each tube. The samples are then shaked for 30 min at 4° C., followed by centrifugation at 14,000 rpm (20,800×g) for 5 min at ambient temperature. The supernatants are transferred to fresh 1.5 ml tubes. 5 μl containing 1 μg of the HMGI-C specific antibody are added to one tube, and both tubes are incubated over night on the rotator at 4° C.

On the following day, for each sample 50 μl Protein A/G PLUS-Agarose are mixed with 3 μg salmon sperm DNA and 250 μl 1×PBS by rotation for 30 min at 4° C. The agarose/salmon sperm DNA mix is added to the sample and the no-antibody control and rotated for 2 h at 4° C. The agarose beads are collected by centrifugation at 12,000 rpm (15,300×g) for 1 min, the supernatants are discarded and the tubes are placed on ice. To wash the agarose beads, 1 ml Lysis High Salt Buffer is added to each tube, and the tubes are incubated for 2 min at room temperature on the rotator. After centrifugation for 1 min at 12,000 rpm (15,300×g) the supernatants are discarded. This washing step is repeated once, then the beads are washed twice with 1 ml of wash buffer for 2 min on the rotator. After centrifugation at 12,000 rpm (15,300×g) for 1 min, the supernatants are discarded and the agarose beads are resuspended in 150 μl 1% SDS solution. The input control is thawed and reintegrated into the following steps. The samples are incubated in a shaking waterbath at 65° C. for 2 h to reverse the cross-links, centrifuged at 12,000 rpm (15,300×g) for 3 min, and incubated over night in the shaking waterbath at 65° C.

To remove residual agarose beads, the samples are centrifuged at 12,000 rpm (15,300×g) for 3 min and the supernatants transferred to new 2.0 ml tubes. They are then diluted 1:2 with water to avoid precipitation, and 5 volumes of buffer PBI (Qiagen PCR Purification Kit) are added to each sample. 700 μl of each sample is applied to QIAquick Spin Columns (Qiagen) and centrifuged for 1 min at 13,000 rpm (17,900×g). This step is repeated until the samples are applied to the columns completely. The columns are washed with 700 μl buffer PE and centrifuged at 13,000 rpm (17,900×g) for 1 min, the flow through is discarded, and the columns are re-centrifuged to remove residual buffer. To elute the DNA, the columns are transferred to new 1.5 ml tubes and 40 μl water is applied to the membrane of the columns. Following incubation at room temperature for 1 min, the columns are centrifuged at 13,000 rpm (17,900×g) for 1 min. The flow through is applied to the membrane of the columns to increase the final DNA concentration, incubated for 1 min at room temperature, and the columns are centrifuged again at 13,000 rpm (17,900×g) for 1 min. Finally, the eluted DNA is immediately used for RT-PCR or stored at −20° C.

### EXAMPLE 5

### Immunoprecipitation of HMG-DNA Complexes

Besides the methods outlined in the previous examples a further method outlined below can be used for immunopreciptation of HMG-DNA complexes using antibodies directed against proteins of the HMGA and the HMGB family.

### Chromatin Immunoprecipitation (ChIP)

Amniotic fluid was centrifuged at 1000×g for 10 min, and 2 ml of the supernatant were transferred to a 13 ml tube. To cross-link the protein DNA complexes, 37% formaldehyde was added to a final concentration of 1%. Cross-linking was performed between two and six hours, respectively, after amniocentesis. After 10 min of incubation at room temperature, the cross-linking reaction was stopped by adding 1 M glycine to a final concentration of 0.125 mM and incubated for 5 min. The sample was divided by half and transferred to two 1.5 ml tubes, and 50 μl of Dynalbeads Protein G (Invitrogen, Karsruhe, Germany) were added to each tube. The samples were shaken for 30 min at 4° C. and the beads were removed with a magnetic rack at ambient temperature. The supernatants were transferred to fresh 1.5 ml tubes. 10 μl containing 2 μg of the HMGA2 specific antibody (Santa Cruz Biotechnology, Heidelberg, Germany) were added to one tube, and both tubes were incubated overnight on the rotator at 4° C.

On the following day, for each sample 50 μl Dynalbeads Protein G were mixed with 5.5 μg salmon sperm DNA and 250 μl 1×PBS by rotation for 30 min at 4° C. The Dynalbeads/salmon sperm DNA mix was added to the sample and the no-antibody control and rotated for 2 h at 4° C. The Dynalbeads were collected with a magnetic separator, the supernatants were discarded. The following steps were perfomed in the 4° C. room. To wash the Dynabeads, 1 ml Lysis Buffer (Santa Cruz Biotechnology, Heidelberg, Germany) was added to each tube, and the tubes were incubated for 3 min on the rotator. After collecting the beads the supernatants were discarded. This washing step was repeated once, and then the beads were washed four times with 1 ml Lysis buffer High Salt (Santa Cruz Biotechnology, Heidelberg, Germany) for 3 min on the rotator followed by four times washing with respectively 1 ml Wash buffer (Santa Cruz Biotechnology, Heidelberg, Germany). The last washing step is done once with 1 ml TE buffer. After the supernatants were discarded the beads were resuspended in 150 μl 1% SDS solution. The samples were incubated in a shaking waterbath at 65° C. for 2 h to reverse the cross-links, Dynabeads were removed with the magnetic separator and incubated for 2 h with 5 μl Proteinase K in the shaking waterbath at 65° C.

Samples were diluted 1:2 with water to avoid precipitation, and 5 volumes of buffer PBI (Qiagen PCR Purification Kit) were added to each sample. 700 μl of each sample was applied to QIAquick Spin Columns (Qiagen, Hilden, Germany) and centrifuged for 1 min at 13,000 rpm (17,900×g). This step was repeated until the samples were applied to the columns completely. The columns were washed with 700 μl buffer PE and centrifuged at 13,000 rpm (17,900×g) for 1 min, the flow through was discarded, and the columns were re-centrifuged to remove residual buffer. To elute the DNA, the columns were transferred to new 1.5 ml tubes and 40 μl water was applied to the membrane of the columns. After incubation at room temperature for 1 min, the columns were centrifuged at 13,000 rpm (17,900×g) for 1 min. The flow through was applied to the membrane of the columns to increase the final DNA concentration, incubated for 1 min at room temperature, and the columns were centrifuged again at 13,000 rpm (17,900×g) for 1 min. Finally, the eluted DNA was immediately used for real-time PCR or stored at −20° C.

This as well as the methods outlined previously have been successfully used to enrich cell-free DNA from malignant effusions (pleural and ascitic) of patients suffering from lung cancer, breast cancer, and colon cancer), from sputum samples derived from patients suffering from lung cancer as well as from amniotic and serum samples from pregnant women.

Also, it should be noted that in case of differential diagnosis the fact that DNA can be enriched by either of the methods described in the examples outlined here gives evidence for the presence of populations of cells that have undergone malignant transformation and thus as such can be used as a diagnostic parameter. Further analyses of enriched DNA can be performed for example by quantitative PCR:

Real-Time PCR with Genomic DNA

To determine the possible enrichment of DNA resulting from the ChIP experiment, the samples were measured with the Applied Biosystems 7300 Real-Time PCR System (for concentrations and cycling conditions see protokol above). Three microliters of every sample and its respective no-antibody control were used to deteced the GAPDH gene. The sequences for GAPDH were 5′-6-FAM-AAA GAG CTA GGA AGG ACA GGC AAC TTG GC-TAMRA-3′ for the fluorescent probe, 5′-CCC CAC ACA CAT GCA CTT ACC-3′ for the forward primer and 5′-CCT AGT CCC AGG GCT TTG ATT-3′ for the reverse primer (Operon, Cologne, Germany). Results were calculated by substracting the Ct-value of the sample from the corresponding NoAb-control, followed by 2^{(sample-NoAb control)} to evaluate the x-fold higher amount of starting material of the sample applied in the real-time PCR.

### REFERENCES

The complete references which are recited herein and which are incorporated in their entirety, read as follows:
1: Castagnaro A, Marangio E, Verduri A, Chetta A, D'Ippolito R, Del Donno M, Olivieri D, Di Cola G.
Microsatellite analysis of induced sputum DNA in patients with lung cancer in heavy smokers and in healthy subjects.
Exp Lung Res. 2007 August; 33(6):289-301.
2: Chin S F, Teschendorff A E, Marioni J C, Wang Y, Barbosa-Morais N L, Thorne N P, Costa J L, Pinder S E, van de Wiel M A, Green A R, Ellis 10, Porter P L, Tavare S, Brenton J D, Ylstra B, Caldas C.
High-resolution array-CGH and expression profiling identifies a novel genomic subtype of ER negative breast cancer.
Genome Biol. 2007 Oct. 9; 8(10):R215
3: Climent J, Garcia J L, Mao J H, Arsuaga J, Perez-Losada J.
Characterization of breast cancer by array comparative genomic hybridization.
Biochem Cell Biol. 2007 August; 85(4):497-508.
4: Dahl J A, Collas P.
A quick and quantitative chromatin immunoprecipitation assay for small cell samples.
Front Biosci. 2007 Sep. 1; 12:4925-31.
5: Dehan E, Ben-Dor A, Liao W, Lipson D, Frimer H, Rienstein S, Simansky D, Krupsky M, Yaron P, Friedman E, Rechavi G, Perlman M, Aviram-Goldring A, Izraeli S, Bittner M, Yakhini Z, Kaminski N.
Chromosomal aberrations and gene expression profiles in non-small cell lung cancer.
Lung Cancer. 2007 May; 56(2):175-84.
6: Gao H, Falt S, Sandelin A, Gustafsson J A, Dahlman-Wright K.
Genome-wide identification of estrogen receptor {alpha} binding sites in mouse liver.
Mol Endocrinol. 2007 Sep. 27;
7: Haring M, Offermann S, Danker T, Horst I, Peterhaensel C, Stam M.
Chromatin immunoprecipitation: optimization, quantitative analysis and data normalization.
Plant Methods. 2007 Sep. 24; 3(1):11
8: Helmig S, Schneider J.
Oncogene and tumor-suppressor gene products as serum biomarkers in occupational-derived lung cancer.
Expert Rev Mol Diagn. 2007 September; 7(5):555-68.
9: Hsu H S, Chen T P, Hung C H, Wen C K, Lin R K, Lee H C, Wang Y C.
Characterization of a multiple epigenetic marker panel for lung cancer detection and risk assessment in plasma.
Cancer. 2007 Sep. 17;
10: Hwang E S, Bowen P E.
DNA damage, a biomarker of carcinogenesis: its measurement and modulation by diet and environment.
Crit Rev Food Sci Nutr. 2007; 47(1):27-50.
11: Jing F, Zhang J, Tao J, Zhou Y, Jun L, Tang X, Wang Y, Hai H.
Hypermethylation of tumor suppressor genes BRCA1, p16 and 14-3-3sigma in serum of sporadic breast cancer patients.
Onkologie. 2007 February; 30(1-2):14-9.
12: Levenson V V.
Biomarkers for early detection of breast cancer: what, when, and where?
Biochim Biophys Acta. 2007 June; 1770(6):847-56.
13: Maron J L, Johnson K L, Bianchi D W.
Cell-free fetal DNA plasma extraction and real-time polymerase chain reaction quantification.
Methods Mol Med. 2007; 132:51-63.
14: McCann J A, Muro E M, Palmer C, Palidwor G, Porter C J, Andrade-Navarro M A, Rudnicki M A.
ChIP on SNP-chip for genome-wide analysis of human histone H4 hyperacetylation.
BMC Genomics. 2007 Sep. 14; 8(1):322
15: Ollikainen M, Hannelius U, Lindgren C M, Abdel-Rahman W M, Kere J, Peltomaki P.
Mechanisms of inactivation of MLH1 in hereditary nonpolyposis colorectal carcinoma: a novel approach.
Oncogene. 2007 Jul. 5; 26(31):4541-9.
16: Ongenaert M, Van Neste L, De Meyer T, Menschaert G, Bekaert S, Van Criekinge W.
PubMeth: a cancer methylation database combining text-mining and expert annotation.
Nucleic Acids Res. 2007 Oct. 15;
17: Riesterer O, Milas L, Ang K K.
Use of molecular biomarkers for predicting the response to radiotherapy with or without chemotherapy.
J Clin Oncol. 2007 Sep. 10; 25(26):4075-83.
18: Schwarzenbach H, Muller V, Beeger C, Gottberg M, Stahmann N, Pantel K.
A critical evaluation of loss of heterozygosity detected in tumor tissues, blood serum and bone marrow plasma from patients with breast cancer.
Breast Cancer Res. 2007 Oct. 3; 9(5):R66
19: Soussi T, Wiman K G.
Shaping Genetic Alterations in Human Cancer: The p53 Mutation Paradigm.
Cancer Cell. 2007 October; 12(4):303-12.
20: Taback B, Giuliano A E, Lai R, Hansen N, Singer F R, Pantel K, Hoon D S.
Epigenetic analysis of body fluids and tumor tissues: application of a comprehensive molecular assessment for early-stage breast cancer patients.
Ann N Y Acad Sci. 2006 September; 1075:211-21.

The features of the present invention disclosed in the specification, the sequence listing, the claims and/or the drawings may both separately and in any combination thereof be material for realizing the invention in various forms thereof.

1. A method for the detection, separation and/or enrichment of fetal and embryonic cells in a maternal body fluid, comprising
a) contacting a sample of a maternal body fluid containing or presumed to contain fetal and/or embryonic cells with an interaction partner of a high mobility group protein of the HMGA family, whereupon a complex is formed between the interaction partner and the fetal and embryonic cells;
b) detecting the complex; and optionally
c) separating and/or enriching fetal and embryonic cells from the maternal body fluid.

2. (canceled)

3. The method according to claim 1, wherein precipitation or chromatography is used to separate the fetal and embryonic cells from the maternal body fluid sample.

4.-6. (canceled)

7. A method the detection of fetal and/or embryonic cells, or fetal and/or embryonic chromatin and/or fetal and/or embryonic nucleic acid(s) in a maternal body fluid, comprising
a) contacting a maternal body fluid sample with a primer or probe specific for a gene or transcript of such gene encoding a high mobility group protein of the HMGA family, whereupon the primer or probe hybridizes or binds to a gene or transcript contained in the fetal and embryonic cells; and
b) detecting the hybridization or binding of the primer or probe.

8. (canceled)

9. The method according to claim 7, wherein the primer or the probe is introduced into the fetal and/or embryonic cells.

10. The method of claim 1, wherein the maternal body fluid is selected from the group consisting of: urine, blood and transcervical lavage.

11. A method for distinguishing fetal and/or embryonic cells from maternal cells and/or maternal tissue, comprising
contacting a sample comprising both fetal and/or embryonic cells and maternal cells and/or maternal tissue, with an interaction partner of a high mobility group protein of the HMGA family, whereupon a complex is formed between the interaction partner and a high mobility group protein of the HMGA family in the sample or a gene or transcript coding therefore, such that
the embryonic cells or fetal cells exhibit more of the complex compared to the maternal tissue and/or maternal cells, thereby distinguishing fetal and/or embryonic cells from maternal cells and/or maternal tissue.

12. The method according to claim 11, wherein the maternal cells and/or maternal tissue is selected from the group consisting of: maternal placenta tissue and placental stromal cells.

13. The method according to claim 11, wherein the fetal and embryonic cells are from the trophoblast or the cytotrophoblast.

14. The method according to claim 11, wherein the sample obtained by chorionic villi sampling.

15. A method for the detection, separation, isolation and/or enrichment of chromatin and/or nucleic acid(s) from a tumor or cancer cell, comprising
a) contacting a body fluid sample sample from a the subject suspected of having a tumor or cancer, with an interaction partner, whereupon a complex is formed between the interaction partner and a high mobility group protein of the HMGA family in the sample;
b) detecting the complex; and optionally
c) separating, isolating or enriching the complex from the sample.

16. (canceled)

17. The method according to claim 16, wherein precipitation or chromatography is used to separate, isolate or enrich the complex.

18. (canceled)

19. (canceled)

20. The method of claim 15, wherein the body fluid is selected from the group comprising blood, urine, sputum, effusions, lavage, stool and saliva.

21. (canceled)

22. The method of claim 15, wherein the chromatin and/or the nucleic acid is contained in a sample from a subject assumed to suffer from or being at risk to develop a tumor or cancer, whereby the sample is preferably selected from the group comprising urine, blood, serum, transcervical lavage, sputum, pleural an effusions, ascitic effusions, saliva, biopsies and stool.

23. The method of claim 15, wherein the tumor or cancer is selected from the group comprising malignant epithelial cancers, malignant mesenchymal tumors, tumors of endocrine and neuroendocrine origin, leukemias, and lymphomas.

24. The method of claim 15, wherein the tumor or cancer is selected from the group comprising lung cancer, breast cancer, non-small cell lung cancer, colorectal cancer, ovarian cancer, endometrial cancer, prostate cancer, and pancreatic cancer.

25. The method of claim 7, wherein the nucleic acid is selected from the group consisting of comprising DNA, mRNA, pre-mRNA and processed mRNA.

26. (canceled)

27. The method of claim 1, wherein the interaction partner is selected from the group consisting of: antibodies, peptide aptamers, anticalines, aptamers, spiegelmers, promers and probes to the high mobility group protein of the HMGA family or the gene or transcript coding therefore.

28.-60. (canceled)

61. The method of claim 1, wherein a complex is formed between the interaction partner and fetal and/or embryonic chromatin and/or fetal and/or embryonic nucleic acid(s).

62. The method of claim 1, wherein a complex is formed between the interaction partner and fetal and/or embryonic chromatin and/or fetal and/or embryonic nucleic acid(s).

63. The method of claim 7, wherein hybridization of the primer is detected by detecting an amplicon following a polymerase chain reaction.

64. The method of claim 7, wherein the maternal body fluid is selected from the group consisting of: urine, blood and transcervical lavage.

65. The method of claim 15, wherein the nucleic acid is selected from the group consisting of: DNA, mRNA, pre-mRNA and processed mRNA.
